(19) United States
(12) Patent Application Publication
(10) Pub. No.: US 2008/0206338 A1
(43) Pub. Date: Aug. 28, 2008

(54) CONTROLLED RELEASE FORMULATIONS OF AN ALPHA-ADRENERGIC RECEPTOR ANTAGONIST

(76) Inventors: Nagaprasad Vishnubhotla, Andhra Pradesh (IN); Vinodkumar Gurunath Indure, Andhra Pradesh (IN); Sindhuja Balakrishnan, Andhra Pradesh (IN); Sivakumaran Meenakshisunderam, Andhra Pradesh (IN)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/034,414

(22) Filed: Feb. 20, 2008

(30) Foreign Application Priority Data
Feb. 20, 2007 (IN) 332/CHE/2007
Apr. 16, 2007 (IN) 798/CHE/2007

(51) Int. Cl. A61K 9/00; A61K 31/517; A61P 13/00

(52) U.S. Cl. 424/487; 424/486; 514/266.24; 424/488

(57) ABSTRACT

The present invention relates to controlled release formulations comprising an alpha-adrenergic receptor antagonist. More particularly, the present invention relates to controlled release matrix formulation comprising alfuzosin hydrochloride and a combination of hydrophilic and hydrophobic polymers prepared by direct compression.

### CROSS-REFERENCE TO RELATED APPLICATIONS

This application claims benefit of priority under 35 USC § 119 of Indian Application 332/CHE/2007 filed Feb. 20, 2007, and Indian Application 798/CHE/2007, filed Apr. 16, 2007, each of which is incorporated by reference in its entirety.

### FIELD OF THE INVENTION

The present invention relates to controlled release formulations comprising an α-adrenergic receptor antagonist. More particularly, the present invention relates to controlled release matrix formulations comprising alfuzosin hydrochloride and a combination of hydrophilic and hydrophobic polymers prepared by direct compression.

### BACKGROUND OF THE INVENTION

Alfuzosin is a selective α-adrenergic receptor antagonist that belongs to the chemical class of 4-amino-6,7-dimethoxyquinazol-2-yl-alkylene diamines. Chemically, alfuzosin, as its hydrochloride salt, is (R,S)-N-[3-[(4-amino-6,7-dimethoxy-2-quinazolinyl)methylamino]propyl]tetrahydro-2-furancarboxamide hydrochloride and is disclosed in U.S. Pat. No. 4,315,007.

Alfuzosin is commercially available as extended release tablets under the trade name UROXATRAL® in the United States. Alfuzosin is also commercially available as film coated tablets as well as extended release tablets under the trade name XATRAL® in Europe. It is indicated for the symptomatic treatment of benign prostatic hypertrophy (BPH).

Controlled-release dosage forms are characterized in that they convey a markedly larger amount of medicinal product than traditional pharmaceutical preparations, so as to allow the dosage regimen to be simplified. Most of these therapeutic systems are capable of releasing the active substance conveyed per se, at a constant rate up to complete release of the active substance. These systems may be applied widely to administer medicinal products that are absorbed uniformly in the gastrointestinal tract.

Various patents discuss the use of pharmaceutical carriers in the preparation of controlled release formulations. U.S. Pat. No. 4,680,323 describes a sustained release pharmaceutical carrier suitable for admixture with active ingredients comprising: (a) 5.5-98.5% by weight of hydroxypropyl methylcellulose; (b) 0.25-4.5% by weight of hydroxypropyl cellulose; (c) 1-90% by weight of a carboxyvinyl polymer.

Alfuzosin has a short half-life and shows the characteristic of being absorbed preferentially in the upper part of the gastrointestinal tract and, in particular, being absorbed in the duodenum and the jejunum. Controlled release compositions of alfuzosin provide various advantages over conventional multiple dosing including better patient compliance, reduced fluctuations of plasma drug levels, and reduced toxicity.

Bilayer or triple layer gastro-retentive matrix tablets are addressed by Liu et al. International Journal of Pharmaceutics, 2008, 348(1-2), 27-34. Such tablets have drawbacks in that, the tablets are initially heavier than the density of the gastric fluids in which they will be immersed. The tablets may have a risk of being expelled from the stomach before having acquired sufficient flotation power. Further, the manufacture of multi-layer tablets is time consuming, complex to produce, involves special facilities, and is consequently relatively expensive.

U.S. Pat. No. 5,589,190 discusses pharmaceutical compositions containing a core, namely a tablet affording immediate release or sustained release or microparticles affording immediate release of alfuzosin hydrochloride, coated with a membrane whose nature and thickness enable the release of the active principle to be controlled on the basis of pH and time.

EP 0 700 285 discusses drug delivery compositions of alpha adrenoceptor blocking agents that have a biphasic drug release profile, characterized in that the composition is adapted to release the drug in two portions, the first portion of the drug being released in the upper GIT and the second portion of the drug being released by sustained release in the terminal ileum/colon. This patent describes matrix compositions using hydroxypropyl methylcellulose and a coating that is designed to dissolve under the conditions present in the colonic region.

Due to its intense absorption at the duodenum-jejenum level, alfuzosin hydrochloride is a favourable candidate for the production of a pharmaceutical preparation with controlled release in the proximal/upper parts of the gastrointestinal tract. The compositions according to U.S. Pat. No. 5,589,190 and EP 0 700 285 have the disadvantage of having the drug release mainly in the latter part of the small intestine and in the large intestine thereby decreasing the effective absorption of the drug.

U.S. Pat. No. 6,861,072 describes a gastro-retentive, controlled release two or three layered compositions containing a carbon dioxide generating system that enables floatation of the dosage form.

US 2004/0115259 describes a process for the production of a homogeneous monolithic floating tablet which involves homogeneous mixing of active principle with a quantity of excipient from 50.00 to 99.90% of the total mass, the excipient being selected from at least one compound of the family of cellulosic derivatives and/or povidone derivatives and/or derivatives of polyvinyl acetate, and finally compression of this mixture to monolithic homogeneous tablet with immediate flotation in the gastric medium. This publication describes that using specific excipients, immediate floatation can be ensured if a specific compression pressure is used. However, this method has disadvantages in commercial processing such as damaging the tablet surface if it is too friable at the low pressures used for compression to obtain a low-density tablet. Moreover, the low pressures that are normally required for quick floatation may result in altering (quickening) the dissolution such that drug release is not sustained to the required levels.

US 2006/0062845 and US 2006/0062846 describe a composition comprising: a polymeric matrix; and alfuzosin in the polymeric matrix, wherein the composition is monolithic in form, and the polymeric matrix is adapted to release 13-33% of the alfuzosin within 2 hours of administration, 40-60% of the alfuzosin within 7 hours of administration and greater than 80% of the alfuzosin within 20 hours of administration. It is also disclosed a polymeric matrix which is composed of hydrophilic polymer that swell upon contact with gastric fluid.

US 2007/0292505 discusses a matrix tablet comprising alfuzosin dispersed in a controlled release material prepared by wet or dry granulation, wherein the tablet following single dose administration under fed conditions to human subjects exhibits a mean time to maximum plasma concentration of about 3 hours to about 14 hours.

US 2008/0003286 describes a composition comprising multiple reservoirs comprising a coating comprising alfuzosin applied onto a pharmaceutically inert particle, and having an outer hydrophilic or hydrophobic coating, dispersed in an external matrix composition comprising a hydrophilic polymer.

WO 2004/028503 describes a controlled release composition containing an inner core comprising at least one cellulose derivative, and an amphiphilic material and an outer layer comprising polymer and plasticiser. This publication mentions the use of release retarding agents in both the inner core as well as the outer layer. Moreover, the example mentions the use of two polymers (a core containing HPMC and outer coating of ethyl cellulose) that are completely different with respect to their properties. This makes the formulation process complicated and even more difficult to control or predict the in vitro/in vivo release profile.

WO 2004/037228 describes sustained release compositions comprising a functional layer, containing drug and release retarding ingredient such as cellulose polymer, methacrylate polymer, acrylic acid polymer, block copolymer, gum and polyethylene oxide and optionally, one or more nonfunctional layers. In this publication, all the examples given illustrate the usage of a combination of two different hydrophilic polymers for retarding drug release. Moreover, the use of hydrophilic polymers may result in a formulation that would swell and erode, resulting in a constant change in the surface area available for drug release.

WO 2006/094736 discusses a solid controlled-release formulation of alfuzosin hydrochloride, hydrophilic polymers, polyvinylpyrrolidone and lactose.

WO 2006/021692 describes a composition in the form of a gastric resident matrix tablet comprising alfuzosin hydrochloride characterized in that when contacted with a medium representative of the gastric fluid, it increases in volume, after fifteen minutes, by a swelling rate of at least 200%. This patent publication further discloses the gastric resident matrix tablet comprising 30 to 80% by weight of povidone or polyvinyl acetate, 5 to 25% by weight of crospovidone and 5 to 40% by weight of carbomer.

WO 2007/080509 discusses a sustained release formulation comprising alfuzosin and at least one hydrophilic rate controlling polymer in the first and second layers and an optional third layer comprising one or more hydrophilic rate controlling polymers.

EP 1 194 131 describes a delayed release coated core producing a timed pulse release, comprising an active substance in its core and a polymer coating comprising at least one or more ammonio methacrylate copolymers, characterized in that the core comprises at least one or more surfactants.

IN 628/MUM/2005 relates to a gastro retentive sustained release dosage form comprising alfuzosin and hydrophobic polymers, wherein the drug release is by surface erosion.

IN 1419/MUM/2005 relates to a bilayer sustained tablet comprising alfuzosin along with a hydrophilic polymer in the first layer and a hydrophobic polymer in the second layer with or without drug.

Sustained release alfuzosin tablets comprising hydrogenated castor oil and dicalcium phosphate and their effect are disclosed by Hoang et al. Tap Chi Duoc Hoc, 2006, 46(1), 22-26.

Controlled-release matrix tablets of alfuzosin-HCl prepared using low viscous hydroxypropyl methylcellulose (HPMC K-100 and HPMC 15 cps) and its comparison with a commercial product are disclosed in Nair et al. Pharmaceutical Development and Technology, 2007, 12(6), 621-625.

The above prior art references address controlled release formulations of alfuzosin hydrochloride using polymers prepared by dry or wet granulation. However, there still exists a need for developing controlled release formulation of alfuzosin hydrochloride to overcome the disadvantages/drawbacks associated with the prior art. Surprisingly, the controlled release formulations of alfuzosin of the present invention, which release the drug over a prolonged period of time, have less chance of burst release, have good gastroretentive properties, and retain their initial form to a considerable extent.

### OBJECTIVE OF THE INVENTION

Accordingly, the main objective of the present invention is to provide controlled release matrix formulation of alfuzosin hydrochloride.

Yet another objective of the present invention is to provide a controlled release matrix formulation, comprising hydrophilic and hydrophobic polymers prepared by simple formulation process as compared to the complex bilayer or tri-layer tablets disclosed in prior art.

Yet another objective of the present invention is to provide controlled release matrix formulation of alfuzosin hydrochloride in such a way that it will comply with the reference product in terms of in vivo parameters for bioequivalence such as Cₘₐₓ, AUC, Tₘₐₓ and in vitro parameters like dissolution and disintegration.

### SUMMARY OF THE INVENTION

Accordingly, the present invention provides a controlled release matrix formulation comprising alfuzosin hydrochloride and a combination of hydrophilic and hydrophobic polymers prepared by direct compression.

### DETAILED DESCRIPTION OF THE INVENTION

The most commonly used method of modulating the drug release is to include it in a matrix system. Hydrophilic polymer matrix systems are widely used in oral controlled drug delivery because of their flexibility to obtain a desirable drug release profile, cost-effectiveness, and broad regulatory acceptance. The drug release for extended duration, particularly for highly water-soluble drugs, using a hydrophilic matrix system is restricted due to rapid diffusion of the dissolved drug through the hydrophilic gel network. For such drugs with high water solubility, hydrophobic polymers are suitable as matrixing agents for developing sustained-release dosage forms. Hydrophobic polymers provide several advantages, ranging from good stability at varying pH values and moisture levels. Faster release of the drug from the hydrophilic matrix is probably due to faster dissolution of the highly water-soluble drug from the core and its diffusion out of the matrix forming the pores for entry of solvent molecules. Incorporation of a hydrophobic polymer in addition to a hydrophilic polymer, controls the drug release to some extent, which could be attributed to the decreased penetration of the solvent molecules in the presence of a hydrophobic polymer leading to decreased diffusion of the drug from the matrix.

Suitable hydrophilic polymers used according to the present invention include polyvinylpyrrolidone, hydroxypropyl cellulose, hydroxypropylmethyl cellulose, methylcellulose, vinyl acetate copolymers, polysaccharides such as alginate, xanthum gum and the like, polyethylene oxide, acrylic acid copolymers such as carbomer; maleic anhydride/methyl vinyl ether copolymers and derivatives and mixtures thereof. The hydrophilic polymers may be used in the range of about 20% to about 70% by weight of the composition.

Suitable hydrophobic polymers include wax materials, cellulose derivatives such as ethyl cellulose or cellulose acetate, copolymers of polyvinyl alcohol and high molecular weight polyvinyl alcohols. The hydrophobic polymers may be used in the range of about 8% to about 70% by weight of the composition.

Suitable wax materials used in accordance with the present invention are selected from hydrogenated vegetable oil, carnauba wax, candellia wax, beeswax, paraffin wax, stearic acid, glyceryl behenate, cetyl alcohol, cetostearyl alcohol, and mixtures thereof.

In another embodiment of the present invention, the controlled release formulation further comprises one or more excipients selected from diluents, binders, glidants and lubricants.

Suitable diluents of the present invention are selected from water insoluble diluents such as calcium phosphate-dibasic, calcium silicate and water soluble diluents such as microcrystalline cellulose, lactose, sucrose, starch, polyols such as mannitol, sorbitol, xylitol, maltitol and the like and mixtures thereof. The diluent may be used in the range of about 15% to about 40% by weight of the composition.

Suitable binders of the present invention are selected from hydroxypropyl cellulose, hydroxypropylmethyl cellulose, starch, povidone, copolyvidone, microcrystalline cellulose, pregelatinized starch and the like and mixtures thereof. The binder may be used in the range of about 5% to about 20% by weight of the composition.

Suitable lubricants of the present invention are selected from sodium stearyl fumarate, magnesium stearate, hydrogenated vegetable oil, stearic acid, calcium stearate, glyceryl behenate, sodium lauryl sulfate, talc and the like and mixtures thereof. The lubricant may be used in the range of about 0.5% to about 1.5% by weight of the composition.

Suitable glidants include talc, colloidal silicon dioxide, corn starch and the like. The glidant may be used in the range of about 0.5% to about 1.5% by weight of the composition.

The tablets may be uncoated or optionally coated with film coating composition. The coating solution mainly comprises of film forming polymers and one or more of plasticizers, opacifiers and the like.

Suitable film forming polymers used according to the present invention is selected from hydroxypropylmethyl cellulose, hydroxypropyl cellulose hydroxyethyl cellulose and the like.

A suitable opacifier used according to the present invention is titanium dioxide.

In a preferred embodiment of the present invention, the controlled release matrix formulation comprises alfuzosin hydrochloride, about 20% to about 70% by weight of hydrophilic polymer selected from hydroxypropyl methylcellulose, hydroxypropyl cellulose, acrylic acid copolymers such as carbomer, povidone or a mixture thereof; about 8% to about 70% by weight of hydrophobic polymer selected from hydrogenated vegetable oil, glyceryl behenate and ethyl cellulose; about 15% to about 40% by weight of diluent selected from dibasic calcium phosphate, lactose and microcrystalline cellulose; about 5% to about 20% by weight of binder selected from povidone, hydroxypropyl methylcellulose and starch; about 0.5% to about 1.5% by weight of lubricant selected from magnesium stearate, sodium stearyl fumarate and stearic acid; about 0.5% to about 1.5% by weight of glidant selected from talc, colloidal silicon dioxide and corn starch prepared by direct compression.

In another embodiment of the present invention the controlled release matrix formulation comprising alfuzosin hydrochloride and a combination of hydrophilic and hydrophobic polymers is prepared by direct compression process, which comprises the steps of

i) blending alfuzosin hydrochloride, hydrophilic and hydrophobic polymers with one or more pharmaceutical acceptable excipient,
ii) lubricating the blend of step (i),
iii) compressing the lubricated blend to obtain tablets and
iv) optionally coating the core tablets with film forming materials.

In another embodiment, there is provided a method of treating benign prostatic hyperplasia (BPH) comprising administering a controlled release matrix formulation of alfuzosin prepared according to the present invention.

The following examples are provided to further exemplify the invention and are not intended to limit the scope of the invention. It is obvious to those skilled in the art to find out the composition for other dosage forms and substitute the equivalent excipients as described in this specification or with the one known to the industry.

### EXAMPLE 1

| S. No. | Ingredients | Quantity in mg |
|---|---|---|
| 1 | Alfuzosin hydrochloride | 10 |
| 2 | Hypromellose | 136.96 |
| 3 | Povidone | 35 |
| 4 | Ethyl cellulose | 70 |
| 5 | Dibasic calcium phosphate | 91.04 |
| 6 | Colloidal silicon dioxide | 3.5 |
| 7 | Magnesium stearate | 3.5 |
| | Core Tablet Weight | 350 |

The processing steps involved in manufacturing alfuzosin hydrochloride tablets are given below:
1. blending alfuzosin HCl and colloidal silicon dioxide,
2. mixing the blend of step (1) with hypromellose, povidone and ethyl cellulose,
3. mixing the blend of step (2) with dibasic calcium phosphate,
4. lubricating the blend of step (3) with magnesium stearate,
5. compressing the blend of step (4) into a tablet, and
6. optionally coating the tablets obtained in step (5) with film coating materials.

The compositions given in Examples 2 to 10 were prepared using the similar procedure described in Example 1.

### EXAMPLE 2

| S. No. | Ingredients | Quantity in mg |
|---|---|---|
| 1 | Alfuzosin hydrochloride | 10 |
| 2 | Hypromellose | 122.5 |
| 3 | Povidone | 35 |
| 4 | Hydrogenated vegetable oil | 70 |
| 5 | Carbomer | 5.25 |
| 6 | Dibasic calcium phosphate | 100.25 |
| 7 | Colloidal silicon dioxide | 3.5 |
| 8 | Magnesium stearate | 3.5 |
| | Core Tablet Weight | 350 |

### EXAMPLE 3

| S. No. | Ingredients | Quantity in mg |
|---|---|---|
| 1 | Alfuzosin hydrochloride | 10 |
| 2 | Hydrogenated vegetable oil | 210 |
| 3 | Povidone | 35 |
| 4 | Lactose | 91.5 |
| 5 | Magnesium stearate | 3.5 |
| | Core Tablet Weight | 350 |

### EXAMPLE 4

| S. No. | Ingredients | Quantity in mg |
|---|---|---|
| 1 | Alfuzosin hydrochloride | 10 |
| 2 | Hypromellose | 70 |
| 3 | Povidone | 35 |
| 4 | Hydrogenated vegetable oil | 136.96 |
| 5 | Dibasic calcium phosphate | 91.04 |
| 6 | Colloidal silicon dioxide | 3.5 |
| 7 | Magnesium stearate | 3.5 |
| | Core Tablet Weight | 350 |

### EXAMPLE 5

| S. No. | Ingredients | Quantity in mg |
|---|---|---|
| 1 | Alfuzosin hydrochloride | 10 |
| 2 | Hypromellose | 210 |
| 3 | Povidone | 17.5 |
| 4 | Hydrogenated vegetable oil | 87.9 |
| 5 | Dibasic calcium phosphate | 17.6 |
| 6 | Colloidal silicon dioxide | 3.5 |
| 7 | Magnesium stearate | 3.5 |
| | Core Tablet Weight | 350 |

### EXAMPLE 6

| S. No. | Ingredients | Quantity in mg |
|---|---|---|
| 1 | Alfuzosin hydrochloride | 10 |
| 2 | Hypromellose | 175 |
| 3 | Povidone | 17.5 |
| 4 | Hydrogenated vegetable oil | 123 |
| 5 | Dibasic calcium phosphate | 17.5 |
| 6 | Colloidal silicon dioxide | 3.5 |
| 7 | Magnesium stearate | 3.5 |
| | Core Tablet Weight | 350 |

### EXAMPLE 7

| S. No. | Ingredients | Quantity in mg |
|---|---|---|
| 1 | Alfuzosin hydrochloride | 10 |
| 2 | Hypromellose | 157.5 |
| 3 | Povidone | 35 |
| 4 | Hydrogenated vegetable oil | 70 |
| 5 | Dibasic calcium phosphate | 70.5 |
| 6 | Colloidal silicon dioxide | 3.5 |
| 7 | Magnesium stearate | 3.5 |
| | Core Tablet Weight | 350 |

### EXAMPLE 8

| S. No. | Ingredients | Quantity in mg |
|---|---|---|
| 1 | Alfuzosin hydrochloride | 10 |
| 2 | Hypromellose | 164.5 |
| 3 | Povidone | 17.5 |
| 4 | Hydrogenated vegetable oil | 35 |
| 5 | Dibasic calcium phosphate | 116 |
| 6 | Colloidal silicon dioxide | 3.5 |
| 7 | Magnesium stearate | 3.5 |
| | Core Tablet Weight | 350 |

### EXAMPLE 9

| S. No. | Ingredients | Quantity in mg |
|---|---|---|
| 1 | Alfuzosin hydrochloride | 10 |
| 2 | Hypromellose | 157.5 |
| 3 | Povidone | 35 |
| 4 | Hydrogenated vegetable oil | 35 |
| 5 | Dibasic calcium phosphate | 105.5 |
| 6 | Colloidal silicon dioxide | 3.5 |
| 7 | Magnesium stearate | 3.5 |
| | Core Tablet Weight | 350 |

### EXAMPLE 10

| S. No. | Ingredients | Quantity in mg |
|---|---|---|
| 1 | Alfuzosin hydrochloride | 10 |
| 2 | Hydrogenated vegetable oil | 70 |
| 3 | Povidone | 35 |
| 4 | Dibasic calcium phosphate | 228 |
| 5 | Colloidal silicon dioxide | 3.5 |
| 6 | Magnesium stearate | 3.5 |
| | Core Tablet Weight | 350 |

All publications, patents and patent applications discussed herein are incorporated herein by reference in their entirety.

We claim:

1. A single layer controlled release matrix formulation comprising alfuzosin hydrochloride and a combination of hydrophilic and hydrophobic polymers prepared by direct compression.

2. The controlled release matrix formulation of claim 1, wherein the hydrophilic polymer is used in the range of 20-70% by weight of the composition and the hydrophobic polymer is used in the range of 8-70% by weight of the composition.

3. The controlled release matrix formulation of claim 1, wherein the hydrophilic polymer is selected from the group consisting of polyvinylpyrrolidone, hydroxypropyl cellulose, hydroxypropylmethyl cellulose, methyl cellulose, vinyl acetate copolymers, polysaccharides, polyethylene oxide, acrylic acid copolymers such as carbomer; maleic anhydride/methyl vinyl ether copolymers and derivatives and mixtures thereof.

4. The controlled release matrix formulation of claim 1, wherein the hydrophobic polymer is selected from the group consisting of wax materials, cellulose derivatives such as ethyl cellulose or cellulose acetate, copolymers of polyvinyl alcohol and mixtures thereof.

5. The controlled release matrix formulation of claim 4, wherein the wax material is selected from the group of hydrogenated vegetable oil, carnauba wax, candellia wax, beeswax, paraffin wax, stearic acid, glyceryl behenate, cetyl alcohol, cetostearyl alcohol, and mixtures thereof.

6. The controlled release matrix formulation of claim 1, further comprising one or more pharmaceutically acceptable excipients such as diluents, binders, lubricant and glidants.

7. The controlled release matrix formulation of claim 6, wherein the diluent is selected from water insoluble diluents such as calcium phosphate-dibasic, calcium silicate and water soluble diluents such as microcrystalline cellulose, lactose, sucrose, starch, polyols and mixtures thereof.

8. The controlled release matrix formulation of claim 6, wherein the binder is selected from the group of hydroxypropyl cellulose, hydroxypropylmethyl cellulose, starch, povidone, copolyvidone, microcrystalline cellulose, pregelatinized starch and mixtures thereof.

9. The controlled release matrix formulation of claim 6, wherein the lubricant is selected from sodium stearyl fumarate, magnesium stearate, hydrogenated vegetable oil, stearic acid, calcium stearate, glyceryl behenate, sodium lauryl sulfate, talc and mixtures thereof.

10. The controlled release matrix formulation of claim 6, wherein the glidant is selected from talc, colloidal silicon dioxide and corn starch.

11. A single layer controlled release matrix formulation comprising alfuzosin hydrochloride, 20-70% by weight of hydrophilic polymer selected from hydroxypropyl methylcellulose, hydroxypropyl cellulose, acrylic acid copolymers such as carbomer, povidone or a mixture thereof; 8-70% by weight of hydrophobic polymer selected from hydrogenated vegetable oil, glyceryl behenate and ethyl cellulose; 15-40% by weight of diluent selected from dibasic calcium phosphate, lactose and microcrystalline cellulose; 5-20% by weight of binder selected from povidone, hydroxypropyl methylcellulose and starch; 0.5-1.5% by weight of lubricant selected from magnesium stearate, sodium stearyl fumarate and stearic acid; 0.5-1.5% by weight of glidant selected from talc, colloidal silicon dioxide and corn starch prepared by direct compression.
